(19) United States
(12) Patent Application Publication
(10) Pub. No.: US 2013/0260431 A1
(43) Pub. Date: Oct. 3, 2013

(54) Process for the Hydrothermal Carbonization of Biological Material and Use of the Obtained Water for Fermentation

(71) Applicant: Ingo Bauer, Frankfurt am Main (DE)

(72) Inventor: Ingo Bauer, Frankfurt am Main (DE)

(73) Assignee: L'Air Liquide, Societe Anonyme Pour L'Etude et L'Exploitation des Procedes Georges Claude, Paris (FR)

(21) Appl. No.: 13/988,280

(22) PCT Filed: Nov. 14, 2011
(86) PCT No.: PCT/EP2011/070035

§ 371 Date: Jun. 10, 2013

(30) Foreign Application Priority Data
Nov. 18, 2010 (DE) 10 2010 060 656.1

(51) Int. Cl. C12P 7/10

(52) U.S. Cl. CPC C12P 7/10
USPC 435/165; 435/290.4

(57) ABSTRACT

The present invention relates to a process for the hydrothermal carbonization of biological material and to the use of the obtained process water for fermentation. In a first process stage, biomass is obtained after a fermentation process with subsequent distillation and/or rectification, and in a second process stage this biomass of the first process stage is converted into a high-carbon product by means of hydrothermal carbonization, wherein the obtained process water of the second process stage is again supplied to the first process stage.

The present invention relates to a process for the hydrothermal carbonization of biological material and to the use of the obtained process water for fermentation.

The large-scale processing of biological material for obtaining various sugars and/or their secondary products produced by fermentation, such as alcohols and organic acids, currently is performed on an industrial scale.

To obtain the desired products from the biological material, the sugar contained therein is chemically/catalytically or enzymatically converted.

Beside the desired secondary products of these processes by-products are also obtained, which substantially contain all naturally occurring groups of substances which are also contained in the respective starting raw material.

The biomass obtained after a fermentation process with subsequent distillation or rectification is referred to as stillage, as is the case for example in the distillation of alcohol for fuel recovery or of spirits such as whisky. If grain is used as vegetable starting material, reference is made to distiller's grains. The stillage on the one hand contains the proteins and non-sugar-containing carbons of the vegetable starting material as well as the constituents of the biocatalyst used in the fermentation, such as enzymes, bacterial or fungal cultures. The microorganisms used as biocatalyst, such as bacteria and/or fungi, synthesize metabolic products which likewise remain in the stillage.

For the fermentation process, the microorganisms used require a sufficient amount of nutrients for the synthesis of cell proteins, in order to achieve a sufficient cell division and thus a high cell concentration during the fermentation process, which in turn ensures a high volume/time turnover in the fermenter. These nutrients, in particular the nitrogen-containing nutrients, must be supplied to the fermentation process mostly in the form of ammonia, ammonium salts, urea, proteolytic enzyme preparations and/or amino acids. This represents an additional cost factor.

Due to expensive and cost-intensive drying processes, the stillage can be used as animal feed. In wet form, it can be admixed to animal feed. However, not every vegetable starting material is suitable for this purpose.

Some animals, for example, do not feed on rye stillage. Another problem of wet stillage consists in the fact that the same must be utilized soon, in order to avoid rotting processes. In addition, stillage can be used as fertilizer or as substrate for biogas plants.

It is known that vegetable biomass can be converted into a high-carbon product by means of hydrothermal carbonization (HTC). The hydrothermal treatment of plant material was first described in 1913 by Friedrich Bergius. Due to the increasing demand for coal-like products, this approach has been developed further over the years.

The patent specification DE 102007012112 B3 for example describes an apparatus and a process for the hydrothermal carbonization of biomass. However, a prepared biomass must be available for this process, and in the course of the HTC process this biomass is brought to a low pH value and is pressurized. In addition, an amount of process water is obtained, which cannot be reused without further measures.

One possibility of reusing HTC process water is described in the “Offenlegungsschrift” EP 2206688 A1. There, the obtained process water is thermo-chemically processed, so that it can subsequently be introduced into the waste water. A special treatment of the obtained process water, however, is time-, cost- and energy-intensive.

It was the object of the present invention to find an expedient and inexpensive use of the biomass which is obtained in a fermentation process with subsequent distillation. In addition, it was the object to find a possibility to reduce the costs of the supply of nutrients, in particular of nitrogen-containing material, to a fermentation process. Furthermore, it was the object of the present invention to reduce the supply of fresh water and the amount of waste water in a fermentation process.

This object is solved by the process according to the invention for the hydrothermal carbonization of biological material, wherein in a first process stage after a fermentation process with subsequent distillation and/or rectification biomass is obtained, and in a second process stage this biomass of the first process stage is converted into a high-carbon product by means of hydrothermal carbonization, wherein the obtained process water of the second process stage is again supplied to the first process stage.

As biological material any biological material can be used, such as vegetable material, animal material, fungi and bacteria as well as any material originating therefrom. Preferably, there is used a material which contains polysaccharides and/or lignin, in order to obtain a high-carbon product. To obtain a process water rich in nitrogen, it is preferred when the material to be processed contains proteins.

In the process according to the invention it is preferred that in the fermentation process at least the following process steps
mash preparation
fermentation
distillation and/or rectification are carried out, and that the biomass obtained from this fermentation process is hydrothermally carbonized in a second process step, wherein
the high-carbon product obtained is separated and
the process water again is supplied to the mash preparation.

The use of stillage from a fermentation process as starting material for an HTC process has the advantage that waste biomass, which cannot be used as animal feed, need not be disposed of. In addition, the advantage is obtained that the stillage need not be dried for further use, which is energy-saving and hence more economic. Another possibility of saving energy results from the possibility of heat coupling of the HTC reaction heat with the first process stage.

The carbohydrates still contained in the stillage are converted into a water-insoluble form and can very easily be separated from the HTC process water in a separation step succeeding the HTC process. The conversion of the stillage in an HTC process into a high-carbon product (such as HTC coal and/or humus) offers the advantage that it can either be dumped in an environmentally and CO, neutral manner, for example to improve the soil structure, or can also be used as valuable material for chemical-technical processes, such as gasification, combustion and/or direct hydrogenation.

Furthermore, it is part of the process according to the invention that the process water obtained from the HTC process contains nutrients such as amino acids and/or ammonium nitrogen.

During a hydrothermal conversion like that in an HTC process, proteins are converted to water-soluble, low-molecular hydrolysates (amino acids). This behavior of proteins is known (Near critical and supercritical water. Part I. Hydrolytic and hydrothermal processes; G. Brunner; J. of super-critical fluids; 47 373-381, 2009).

In the process according to the invention this property of proteins is utilized in an advantageous way, in that the stillage rich in proteins is subjected to an HTC process. Under energetic reaction conditions, the amino acids formed in this way can be decomposed by partly mineralizing and releasing ammonium nitrogen. Due to the acidic pH value of below 6 during the HTC process, the ammonium nitrogen likewise remains in the HTC process water in dissolved form. After separation of the high-carbon HTC product, the amino acids contained in the process water as well as the ammonium nitrogen remain in the process water and can be supplied to a fermentation process as fraction rich in nutrients.

In this way, savings can be achieved when adding nutrients during fermentation processes. Especially the nitrogen mostly is a limiting factor in metabolic processes and also in the fermentation. Therefore, nitrogen-containing nutrients must be obtained from external sources and be supplied to the fermentation process. By using the HTC process water rich in nutrients for the fermentation process, this additional supply of nutrients, in particular of nitrogen, is not necessary or can at least strongly be reduced. In addition costs are saved, since the amount of fresh water required for the first process stage is reduced by the addition of the process water from the second process stage. Furthermore, the amount of waste water and the waste water load are reduced, which is both cost-saving and environmentally conscious. In particular, these advantages apply to the combination of a process for producing bioethanol with an HTC process.

Furthermore, in the process according to the invention the biomass obtained is heated in the second process stage at a temperature of 120° C. to 350° C., preferably 160° C. to 280° C., particularly preferably 180° C. to 250° C. By heating the stillage in a closed tank, for example in an autoclave, the corresponding water pressure is obtained automatically. It has surprisingly been found that due to the preferred temperature range the formation of phenols from lignocellulose-containing fiber constituents of the stillage is reduced. This advantage provides for an expedient use of the HTC process water, since phenols have an inhibiting effect on the growth of microorganisms and thus can have a disturbing effect in a fermentative process. Therefore, a low concentration of phenols in conjunction with the nutrients contained in the HTC process water has an advantageous effect on a fermentation process.

Furthermore, it is preferred that heating the biomass obtained is effected over a period of 1 min to 12 hours, preferably 5 min to 6 hours.

In a preferred embodiment of the process according to the invention combustible gases, for example synthesis gas, are obtained from the high-carbon product in a further post-processing stage.

Furthermore, it is subject-matter of the process according to the invention that at a pH value ≧5 of the biomass obtained a catalyst is added in the form of an acid, with citric acid being preferred as catalyst. The initial pH value of the stillage mostly lies in the slightly acid range (pH 3.5-5).

Accordingly, the addition of a catalyst in the form of an acid only is required when the pH value should lie above 5.

In one aspect of the process according to the invention all types of grain and/or sugar cane and/or sugar beet and/or manioc and/or other plant materials containing cellulose are used for preparing mash. The types of grain include e.g. wheat, rye, corn, rice, oats, millet, and barley.

In one embodiment of the process according to the invention, the obtained biomass distiller's grains originates from a process for producing bioethanol.

The invention also relates to an apparatus for carrying out the process according to the invention for the hydrothermal carbonization of biological material, wherein the apparatus at least comprises
a reactor for preparing mash
a reactor for fermentation
a distillery with discharge devices for the distillate
a reactor for the HTC process
a separation device with discharge devices for the solid, high-carbon product from the HTC process
a connecting line from the separation device to the reactor for preparing mash, in which the HTC process water is transported.

Furthermore, it is advantageous to use the nutrient-containing process water of a hydrothermal carbonization by the process according to the invention as addition to the mash preparation in a fermentation process.

The following Figures are provided to illustrate the invention and should by no means be construed as limiting the invention.

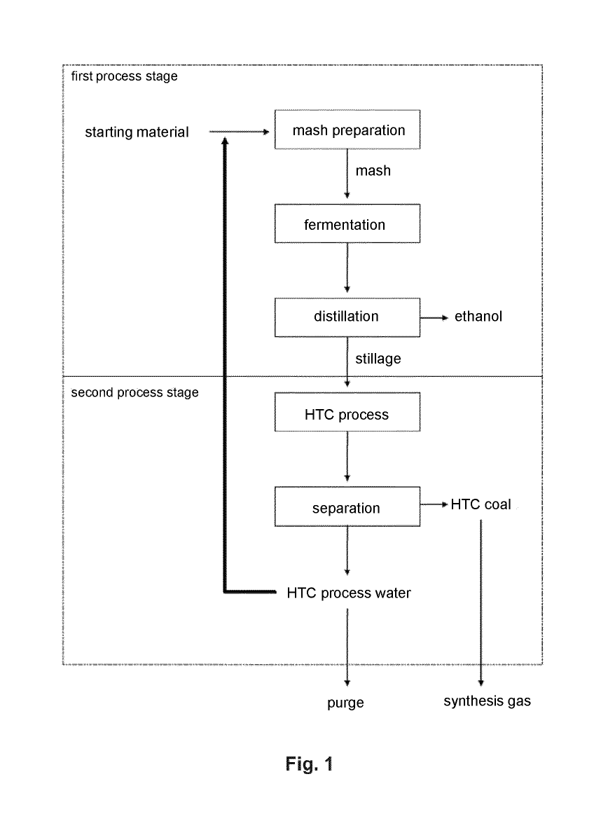

FIG. 1 shows a schematic overview of the most important stages of the process according to the invention, explained by way of example with reference to an integration of the HTC process into a bioethanol process.

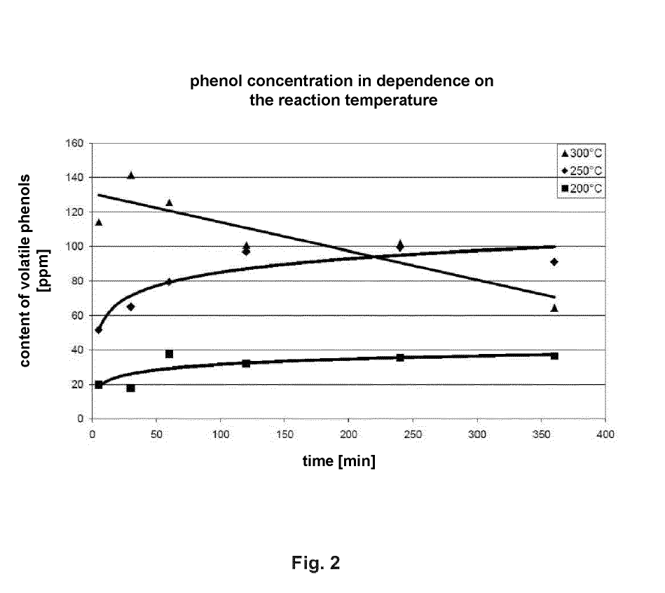

FIG. 2 shows the course of the total phenol content in dependence on the temperature.

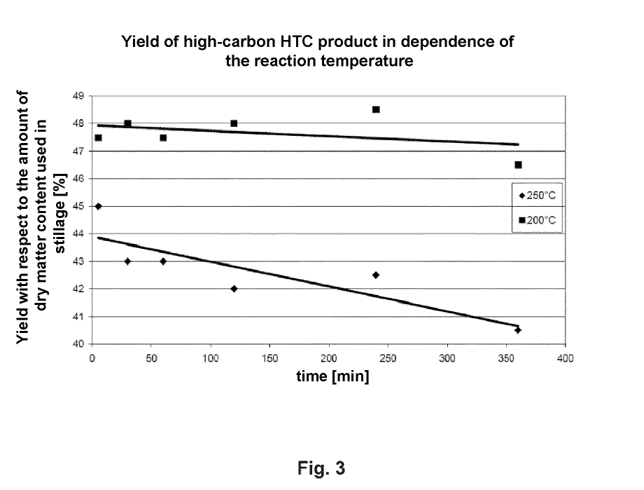

FIG. 3 shows the course of the HTC product yield in dependence on the temperature.

FIG. 1 shows a schematic overview of the most important stages of the process according to the invention. The starting material of the first process stage, which in this case is composed of grain, fresh water and auxiliaries, is used for preparing mash. Microorganisms convert the fermentable carbohydrates from the vegetable biomass into alcohol. By means of distillation the ethanol is obtained, which can be used as fuel, and the distiller's grains are left. This stillage is used as starting material for the second process stage, the HTC process. Under the influence of temperature and pressure, the stillage is converted into a high-carbon product, the HTC coal. In a subsequent separation step, these water-insoluble substances are removed by filtration. The HTC coal then for example can be converted to synthesis gas in further processes. In the HTC process water amino acids and ammonium nitrogen are left, which then are again supplied to the first process stage as fraction rich in nutrients.

FIG. 2 shows the course of the total phenol content in dependence on the temperature. Due to the preferred treatment of the biomass during the HTC process at a temperature of 180° C. to 250° C., wherein in the lower temperature range of 200° C. a treatment of about 6 hours and in the higher temperature range of 250° C. a treatment of about 30 minutes is advantageous, the formation of phenols is minimized and thus the concentration of phenols in the HTC process water is reduced. Since the phenols have a disturbing effect on the metabolism of microorganisms, the HTC process water with a high phenol concentration would not be suitable for use in a fermentation process.

FIG. 3 shows the course of the HTC product yield in dependence on the temperature. It is found that in a temperature range of 180° C. to 220° C. with a treatment time of up to 6 hours a maximum yield of a high-carbon HTC product is ensured.

The process according to the invention will be explained in detail with reference to two exemplary embodiments with respect to the yield of the HTC product in dependence on the temperature. The following Examples are provided to illustrate the invention and should by no means be construed as limiting the invention.

### Exemplary Embodiment 1

In one exemplary embodiment, 100 g stillage from a bioethanol process, in which corn (dry matter content about 20%, pH 4.5) was used as starting material, were used as feedstock. The experiment was carried out at a temperature of 200° C. and a retention time of 240 min at this temperature in a stirred autoclave.

After removing the reaction mixture by filtration, a brown voluminous solid material was left, which after drying in the drying cabinet had a powdery consistency. The filtrate was clear, with a yellow/brown color, and had a pH value of 3.5. The concentration of soluble total nitrogen according to Kjeldahl was 1.0 wt-%.

The wet filter residue of the HTC product had a weight of 29.1 g. After drying in the drying cabinet at 105° C. the filter residue had a weight of 9.7 g.

### Exemplary Embodiment 2

In a second exemplary embodiment, 100 g stillage from a bioethanol process, in which corn (dry matter content about 20%, pH 4.5) was used as starting material, were used as feedstock. The experiment was carried out at a temperature of 250° C. and a retention time of 60 min at this temperature in a stirred autoclave.

After removing the reaction mixture by filtration, a brown voluminous solid material was left, which after drying in the drying cabinet had a powdery consistency. The filtrate was clear, with a yellow/brown color, and had a pH value of 3.5. The concentration of soluble total nitrogen according to Kjeldahl was 1.3 wt-%.

The wet filter residue of the HTC product had a weight of 15.3 g. After drying in the drying cabinet at 105° C. the filter residue had a weight of 8.6 g.

1. A process for the hydrothermal carbonization of biological material, characterized in that in a first process stage after a fermentation process with subsequent distillation and/or rectification biomass is obtained, and in a second process stage this biomass of the first process stage is converted into a high-carbon product by means of hydrothermal carbonization, wherein the obtained process water of the second process stage is again supplied to the first process stage.

2. The process according to claim 1, wherein the fermentation process comprises the following process steps
mash preparation
fermentation
distillation and/or rectification; and wherein the biomass obtained from this fermentation process is hydrothermally carbonized in a second process step, wherein
the high-carbon product obtained is separated and
the process water again is at least partly supplied to the mash preparation.

3. The process according to claim 1, the process water obtained from the HTC process contains amino acids or ammonium nitrogen.

4. The process according to claim 1 wherein, the biomass is heated at a temperature of 120° C. to 350° C.

5. The process according to claim 4 wherein the biomass is heated over a period of 1 min to 12 hours.

6. The process according to claim 1 wherein high-carbon product combustible gases are obtained in a further post-processing stage.

7. The process according to claim 1 wherein when the biomass has a pH value ≧5, a catalyst is added in the form of an acid.

8. The process according to claim 1 wherein grain and/or sugar cane and/or sugar beet and/or manioc and/or other plant materials containing cellulose are used for preparing mash.

9. The process according to claim 1 wherein the biomass comprises distiller's grains.

10. An apparatus for carrying out a process for the hydrothermal carbonization of biological material according to claim 1 wherein the apparatus at least comprises
a reactor for preparing mash
a reactor for fermentation
a distillery with discharge devices for the distillate
a reactor for the HTC process
a separation device comprising discharge devices for the solid, high-carbon product from the HTC process
a connecting line from the separation device to the reactor for preparing mash, in which the HTC process water is transported.

11. (canceled)
